(19) United States
(12) Patent Application Publication
(10) Pub. No.: US 2003/0162687 A1
(43) Pub. Date: Aug. 28, 2003

(54) Surfactants

(75) Inventors: Kazuhiko Tobita, Kawasaki-shi (JP); Masatoshi Saito, Kawasaki-shi (JP); Yoshinori Yamaguchi, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 10/365,391

(22) Filed: Feb. 13, 2003

(30) Foreign Application Priority Data
Feb. 15, 2002 (JP) 2002-037679

(51) Int. Cl. A61K 7/50

(52) U.S. Cl. 510/499; 510/130

(57) ABSTRACT

A surfactant comprising an N-long-chain acyl amino acid or a salt thereof dried by a spray dryer, which contains 280 mesh pass particles at a ratio of 3% by weight or less, and a surfactant comprising an N-long-chain acyl amino acid or a salt thereof dried by a spray dryer, which does not substantially contain 200 mesh pass particles, preferably 140 mesh pass particles. Also provided are surfactants having improved solubility and flowability, which are used for detergents and the like.

### FIELD OF THE INVENTION

The present invention relates to surfactants used for detergents and the like. More specifically, the invention relates to surfactants having improved solubility and flowability.

### RELATED ART

Surfactants are used as raw material ingredients of various kinds of detergents, cosmetics and the like, and those prepared in a liquid or solid form are used depending on properties or purposes thereof. Powdery surfactants are known as a class of surfactants in a solid form. These surfactants are mainly used as powdery detergents such as facial cleansing powder and powdered soap, or as liquid detergents obtained by dissolution in a liquid such as a body shampoo. Powdery surfactants are required to have properties such that they can be easily produced by using a spray drier and have superior solubility when used as facial cleansing powder or the like, and that they cause no blocking when introduced into package containers from a hopper so as to be easily handled. Therefore, it is desired to develop a powdery surfactant having all of the properties.

As for solid surfactants, many studies have been made from viewpoints of solubility, dusting, flowability, compactification and the like. Known methods include, for example, a preparation method consisting of a combination of the spray drying method and the fluidized bed granulation method (Maesaka et al., Funtai to Kogyo (Powder Science & Engineering), Vol. 21, No. 10, p.32 (1989)), preparation methods utilizing, after a spray drying step, rolling granulation, agitation granulation, extrusion granulation and the like (U.S. Pat. No. 3,886,098, Japanese Patent Unexamined Publication (Kokai) Nos. (Hei)2-232299/1990, (Hei)2-232300/1990, (Hei)2-222498/1990, (Hei)2-222499/1990, 2001-152183, Nakamura M., Funtai to Kogyo, Vol. 28, No. 6, p.63 (1996), Isa, H., Yushi (Oils and Fats), Vol. 48, No. 4, p.77 (1995) and the like), a method utilizing granulators in multiple steps (International Patent Publication in Japanese (Kohyo) No. (Hei)10-506141/1998) and the like.

However, a primary object of each of the above methods is achieve compactification of surfactants by increasing bulk density. Although surfactants obtained have various advantages (such as improved dusting and flowability, no foaming at preparation of facial cleansing foam, and easiness to use as washing soap with ready sinking in a liquid), they have drawbacks such that they are hard to be dissolved on hands when they are used as facial cleansing powder or powdered soap, and that they are hardly caked in preparation of soaps. Further, in the aforementioned methods, various builders and binders are used for granulation, and therefore, a problem arises that the above methods cannot be applied to raw materials, per se. Several methods are also available in which a particular composition is used to improve solubility, regardless of employment or no employment of granulation process. However, such methods also have a problem that they cannot be applied to raw materials, per se. A method is also known in which preparation is performed by using a drum dryer without granulation (Kamei S., “Kagaku Kikai no Riron to Keisan (Theory and Calculation for Chemical Machinery)”, 2nd Edition, p.367, Sangyo Tosho (1975)). However, resulting products are obtained in irregular forms and fragile and thus readily breakable, and therefore, almost unsatisfactory from viewpoints of dusting and flowability.

### DISCLOSURE OF THE INVENTION

An object of the present invention is to provide surfactants, and the object of the present invention is to provide surfactants having improved solubility and flowability. More specifically, the object of the present invention is to provide surfactants having superior solubility and flowability as facial cleansing powder, for example, which successfully avoid degradation of quality caused by applying excess heat and change in a composition caused by addition of water.

The inventor of the present invention conducted various researches to achieve the foregoing object. As a result, the inventor found that a surfactant having superior solubility and flowability as well as suppressed dusting was obtainable, without degradation of quality caused by applying excess heat, by decreasing a ratio of particles in conventional surfactants that have a particle size of not more than a particular size. The present invention was achieved on the basis of these findings.

The present invention thus provides a surfactant comprising an N-long-chain acyl amino acid or a salt thereof dried by a spray dryer, which contains 280 mesh pass particles at a ratio of 3% by weight or less. The present invention also provides a surfactant comprising an N-long-chain acyl amino acid or a salt thereof dried by a spray dryer, which does not substantially contain 200 mesh pass particles, and a surfactant comprising an N-long-chain acyl amino acid or a salt thereof dried by a spray dryer, which does not substantially contain 140 mesh pass particles. According to a preferred embodiment of the aforementioned surfactants, provided is the aforementioned surfactants, which contain 30 mesh pass particles at a ratio of 95% by weight or more. From another aspect, the present invention provides a cosmetic composition for skin and/or hair, which contains any of the aforementioned surfactants.

### PREFERRED EMBODIMENT OF THE INVENTION

A type of the N-long-chain acyl amino acid contained in the surfactants of the present invention is not particularly limited. As the acyl group, for example, a saturated or unsaturated straight or branched-chain fatty acyl group having 8 to 22 carbon atoms can be used. A mixture of two or more kinds of N-long-chain acyl amino acids having acyl groups of different chain lengths may be used as the N-long-chain acyl amino acid. Examples of the acyl group include, for example, 2-ethylhexanoyl group, capryloyl group, caproyl group, lauroyl group, myristoyl group, palmitoyl group, stearoyl group, isostearoyl group, oleoyl group, behenoyl group, cocoyl group, tallow fatty acid acyl group, hydrogenated tallow fatty acid acyl group and the like. However, the acyl group is not limited to these examples. The N-long-chain acyl amino acid can be synthesized by, for example, adding a fatty acid chloride to an alkaline solution of an amino acid. The methods are well known to those skilled in the art, and accordingly, an N-long-chain acyl amino acid can be easily obtained.

A type of an amino acid that constitutes the N-long-chain acyl amino acid is not also particularly limited. Examples include glutamic acid, glycine, alanine, threonine and the like. As the amino acid constituting the N-long-chain acyl amino acid, any mixtures of optical isomers or optical antipodes or racemates may be used.

A type of a salt of the N-long-chain acyl amino acid is also not particularly limited. The salt may be chosen from those ordinarily used in the field of the art. Examples include alkali metal salts such as sodium salts and potassium salts, alkaline earth metal salts such as calcium salts and magnesium salts, metal salts such as aluminum salt and zinc salts, ammonium salts, organic amine salts such as monoethanolamine salts, diethanolamine salts and triethanolamine salts, basic amino acid salts such as arginine salts and lysine salts. For the manufacture of the surfactant of the present invention, two or more kinds of substances selected from the group consisting of N-long-chain acyl amino acids and salts thereof may be used.

The surfactant of the present invention is characterized to contain an N-long-chain acyl amino acid or the salt thereof dried by a spray dryer, wherein a ratio of 280 mesh pass particles is 3% by weight or less. In the specification, a “surfactant dried by a spray dryer” means a surfactant generally in a powdery state, and usually means a surfactant consisting of porous particles, hollow particles and the like. The “280 mesh pass particles” are particles that pass through a sieve of 280 mesh (53 μm), and they are generally particles having a particle diameter of less than 53 μm.

Content of the 280 mesh pass particles can generally be measured according to JIS Z8815 “General procedures for sieve analysis test”. For example, the content can be determined by sieving particles through a sieve having a mesh size of 280 (53 μm) with vibration using a vibrator, and measuring weight of particles passed through the sieve. The measurement can be carried out, for example, after vibration for 5 minutes with an amplitude of 2.0 mm, and a sieve according to the JIS standard (JIS Z 8801-1) can be used as the sieve. A specific example of the sieving method is given in the examples of the specification, and accordingly, those skilled in the art can measure a content of 280 mesh pass particles by referring to the aforementioned JIS general procedures and descriptions of the examples of the specification.

When 3% by weight or more of 280 mesh pass particles are contained, both of solubility and flowability of surfactant may be degraded, and dusting property may also be sometimes degraded. The ratio of 280 mesh pass particles is preferably 2% by weight or less, more preferably 1% by weight or less. Most preferably, the surfactant is substantially free from 280 mesh pass particles.

Further, the surfactant of the present invention according to another embodiment is characterized to comprise an N-long-chain acyl amino acid or a salt thereof dried by a spray dryer, wherein 200 mesh pass particles, preferably 140 mesh pass particles are substantially not contained. A content of 200 mesh pass particles or 140 mesh pass particles can also be measured in the same manner as described above. The content of the particles defined in the specification may include an error in a degree acceptable in this field. Generally, a measurement error of approximately several percents is accepted. The term “substantially not contain” means that the content is less than about 0.5% by weight.

The surfactants of the present invention can be produced by, for example, classifying powdery surfactant obtained by using a spray dryer. Any classification means available in the field of the art can be appropriately employed for the classification. For example, a dry type classification is preferred. More specifically, means such as cyclone, sieve, centrifugal classification and the like can be employed. As sieve classification apparatuses, for example, vibration sieve (Dalton), disk-type vibration sieve (Tokuju Kosakusho) and the like can be used, and as centrifugal classification apparatuses, for example, sieve micron separator (HOSOKAWA MICRON CORP.), turbo screener (Turbo Kogyo) and the like can be used. In addition, particles having a small particle diameter separated by the classification can be redissolved, dried and then used again as a raw material. Alternatively, during the preparation of a powdery surfactant using a spray dryer, it is also possible to appropriately choose conditions such as liquid feeding rate, rotation number of atomizer, and temperature of fed wind so that the ratio of particles of the small diameter can be reduced as low as possible.

A purpose of use of the surfactants of the present invention is not particularly limited. Examples of the use include, for example, body shampoos, hair shampoos, facial cleansing powders, bar soaps and the like. For example, cosmetic compositions for skin and/or hair such as body shampoos and hair shampoos containing the surfactants of the present invention are preferred. Such cosmetic compositions can be easily produced by those skilled in the art in an ordinary manner. If necessary, one or more kinds of additives used for manufacture of cosmetic compositions may be added, and such additives can be appropriately chosen by those skilled in the art depending on desired properties.

### EXAMPLES

The present invention will be explained more specifically with reference to examples. However, the scope of the present invention is not limited to the following examples. The unit of values indicated in the following formulation examples is part by weight.

| Formulation Example 1 (facial cleansing powder) | |
|---|---|
| Sodium N-lauroyl-L-glutamate | 18.0 |
| Sodium N-myristoyl-L-glutamate | 12.0 |
| Talc | 10.0 |
| Mannitol | 20.0 |
| Starch | 39.8 |
| Methylparaben | 0.2 |
| Total | 100.0 |
| Formulation Example 2 (facial cleansing powder) | |
| Sodium N-lauroyl-L-glutamate | 6.0 |
| Sodium N-palmoyl-L-glutamate | 4.0 |
| Sodium cocoyl isethionate | 20.0 |
| Talc | 10.0 |
| Mannitol | 20.0 |
| Starch | 39.8 |
| Methylparaben | 0.2 |
| Total | 100.0 |
| Formulation Example 3 (facial cleansing powder) | |
| Sodium N-stearoyl-L-glutamate | 12.0 |
| Sodium N-myristoyl-L-glutamate | 8.0 |
| Sodium cocoyl isethionate | 10.0 |
| Talc | 10.0 |
| Mannitol | 20.0 |
| Starch | 39.8 |
| Methylparaben | 0.2 |
| Toal | 100.0 |
| Formulation Example 4 (powdered shampoo) | |
| Sodium N-cocoyl-L-glutamate | 23.0 |
| Potassium N-lauroyl-L-glutamate | 7.0 |
| O-[2-Hydroxy-3-(trimethylammonio)propyl]- | 0.2 |
| hydroxyethylcellulose chloride | |
| Talc | 10.0 |
| Sorbitol | 20.0 |
| Starch | 39.5 |
| Allantoin | 0.1 |
| Methylparaben | 0.2 |
| Total | 100.0 |
| Formulation Example 5 (facial cleansing powder) | |
| Sodium N-cocoyl glycinate | 12.0 |
| Sodium N-myristoyl-L-glutamate | 10.0 |
| Sodium cocoyl isethionate | 8.0 |
| Talc | 10.0 |
| Mannitol | 20.0 |
| Starch | 39.8 |
| Methylparaben | 0.2 |
| Total | 100.0 |

### Example 1

In the following example, sieve classification was carried out by vibrating a sieve for 5 minutes at an amplitude of 2.0 mm using a vibrator produced by Rctsch. Powdery samples were prepared in a conventional manner by using a spray dryer with sodium N-lauroyl-L-glutamate, sodium N-myristoyl-L-glutamate, sodium N-palmoyl-L-glutamate, and sodium N-cocoyl glycinate as salts of N-long-chain acyl amino acids. Drying was performed under the following conditions.

Spray Drying Conditions

Fed liquid temperature: 50 to 70° C.

Fed liquid concentration: 25 to 31%

Supplied gas temperature: 120 to 150° C.

Exhausted gas temperature: 80 to 100° C.

Atomizer rotation number: 7000 to 9000 r.p.m.

These powdery samples were found to contain 4.7% by weight of 280 mesh pass particles (particles having a particle diameter of less than 53 μm).

From the samples dried by the spray drier, 280 mesh pass cut samples, 200 mesh pass cut samples, and 140 mesh pass cut samples were prepared by using sieves. These samples did not substantially contain particles having a particle diameter under the cut off diameter (53 μm, 75 μm and 106 μm, respectively). Samples that were not subjected to sieve classification were used as controls.

For evaluation of solubility, 0.2 g of each sample to be evaluated was put on hand, added with 2.0 g of tap water and mixed 20 times with fingers, and then the state of the sample was evaluated by visual inspection. The observation was performed by a panel of three experts according to the following evaluation criteria, and an average of evaluation scores of the three experts was calculated. As for the score of evaluation criteria, x represents 1 to less than 2, Δ represents 2 to less than 3, ◯ represents 3 to less than 4, and \mathring{∘} represents 4. The results are shown in Tables 1 to 4.

1: Large aggregations remained.

2: Small undissolved portions remained.

3: Uniformly dissolved.

4: Quickly dissolved.

As for evaluation of flowability, parameters of angle of repose, degree of compaction, spatula angle, and degree of aggregation were measured by using a powder tester Model PT-N produced by HOSOKAWA MICRON CORP. based on the method for evaluating Carr's flowability index. The results of each of the parameters and flowability index are shown in Tables 1 to 4.

TABLE 1
Na lauroylglutamate

| | | | | | |
|---|---|---|---|---|---|
| Particle diameter distribution | ˜53 μm | 4.7% | 0% | 0% | 0% |
| | 53˜75 μm | 7.9% | 8.3% | 0% | 0% |
| | 75˜106 μm | 8.4% | 8.8% | 9.6% | 0% |
| | 106˜μm | 79.0% | 82.9% | 90.4% | 100% |
| Flowability | Angle of repose (°) | 33.0 | 28.6 | 30.6 | 29.1 |
| | Degree of compaction (%) | 18.3 | 14.0 | 12.7 | 13.7 |
| | Spatula angle (°) | 46.7 | 36.7 | 31.7 | 29.5 |
| | Degree of aggregation (%) | 15.1 | 12.5 | 15.7 | 34.6 |
| | Flowability index | 67.0 | 78.0 | 77.0 | 76.0 |
| Solubility | Evaluation of solubility | Δ | ◯ | ⊚ | ⊚ |

TABLE 2
Na myristoylglutamate

| | | | | | |
|---|---|---|---|---|---|
| Particle diameter distribution | ˜53 μm | 7.7% | 0% | 0% | 0% |
| | 53˜75 μm | 6.0% | 6.5% | 0% | 0% |
| | 75˜106 μm | 13.0% | 14.1% | 15.1% | 0% |
| | 106˜μm | 73.3% | 79.4% | 84.9% | 100% |
| Flowability | Angle of repose (°) | 26.6 | 31.1 | 30.1 | 30.8 |
| | Degree of compaction (%) | 16.5 | 12.8 | 11.1 | 11.3 |
| | Spatula angle (°) | 49.3 | 38.8 | 37.0 | 34.8 |
| | Degree of aggregation (%) | 12.3 | 15.9 | 19.9 | 13.5 |
| | Flowability index | 70.0 | 74.5 | 77.5 | 77.0 |
| Solubility | Evaluation of solubility | Δ | ◯ | ⊚ | ⊚ |

TABLE 3
Na palmoylglutamate

| | | | | | |
|---|---|---|---|---|---|
| Particle diameter distribution | ˜53 μm | 5.3% | 0% | 0% | 0% |
| | 53˜75 μm | 4.5% | 4.8% | 0% | 0% |
| | 75˜106 μm | 16.9% | 17.8% | 14.4% | 0% |
| | 106˜μM | 73.3% | 77.4% | 81.3% | 100% |
| Flowability | Angle of repose (°) | 39.3 | 35.5 | 37.4 | 32.0 |
| | Degree of compaction (%) | 15.6 | 12.4 | 12.4 | 10.2 |
| | Spatula angle (°) | 59.0 | 52.4 | 53.4 | 49.2 |
| | Degree of aggregation (%) | 17.6 | 21.5 | 20.6 | 26.4 |
| | Flowability index | 65.5 | 68.5 | 67.0 | 71.5 |
| Solubility | Evaluation of solubility | Δ | ◯ | ⊚ | ⊚ |

TABLE 4
Na cocoylglycinate

| | | | | | |
|---|---|---|---|---|---|
| Particle diameter distribution | ∫53 μm | 5.5% | 0% | 0% | 0% |
| | 53˜75 μm | 4.4% | 6.5% | 0% | 0% |
| | 75˜106 μm | 17.1% | 15.3% | 9.2% | 0% |
| | 106˜μm | 73.0% | 78.2% | 90.8% | 100% |
| Flowability | Angle of repose (°) | 36.7 | 33.0 | 31.0 | 28.6 |
| | Degree of compaction (%) | 16.4 | 14.5 | 15.1 | 15.6 |
| | Spatula angle (°) | 62.0 | 56.0 | 50.0 | 43.4 |
| | Degree of aggregation (%) | 32.8 | 28.4 | 28.1 | 27.9 |
| | Flowability index | 58.0 | 70.0 | 70.0 | 74.0 |
| Solubility | Evaluation of solubility | Δ | ◯ | ⊚ | ⊚ |

The surfactants of the present invention can be produced by a simple method at a low cost, and have superior solubility and flowability as well as suppressed dusting. Therefore, the surfactants can be suitably used for applications of body shampoos, hair shampoos and the like.

What is claimed is:

1. A surfactant comprising an N-long-chain acyl amino acid or a salt thereof dried by a spray dryer, which contains 280 mesh pass particles at a ratio of 3% by weight or less.

2. A surfactant comprising an N-long-chain acyl amino acid or a salt thereof dried by a spray dryer, which does not substantially contain 200 mesh pass particles.

3. A surfactant comprising an N-long-chain acyl amino acid or a salt thereof dried by a spray dryer, which does not substantially contain 140 mesh pass particles.

4. The surfactant according to any one of claims 1 to 3, which contains 30 mesh pass particles at a ratio of 95% by weight or more.

5. A cosmetic composition for skin and/or hair, which comprises the surfactant according to any one of claims 1 to 4.
